(19) United States
(12) Patent Application Publication
(10) Pub. No.: US 2022/0001295 A1
(43) Pub. Date: Jan. 6, 2022

(54) An improved process to increase the performance on filtrate extention

(71) Applicant: PREM ANAND MUKUND Honavar, Honavar (IN)

(72) Inventors: PREM ANAND MUKUND Honavar, Honanvar (IN); SANGEETHA Premanand Honavar, Honavar (IN)

(21) Appl. No.: 17/280,898

(22) PCT Filed: Nov. 4, 2019
(86) PCT No.: PCT/IN2019/000034

§ 371 Date: Mar. 28, 2021

(30) Foreign Application Priority Data
Nov. 19, 2018 (IN) 201841043450

(51) Int. Cl. B01D 9/00; C07B 63/00; B01D 37/00

(52) U.S. Cl. CPC B01D 9/005; B01D 2009/0086; B01D 37/00; C07B 63/00

(57) ABSTRACT

This invention is related to an improvement in the process of filtrate extension of a reaction where at least a set of filtrates of a set of reaction are mixed together to form for a filtrate extension thereby the reaction yields higher order chemical auxiliaries and the reaction is observed that either as convergent or divergent pathway substrate reacts to give qualitative products quantitative, conversions and very less in numbers. Among qualitative conversion reaction filtrate takes pivotal role in synthesizing chemical auxiliaries.

### BACKGROUND

### Technical Field of Invention

This invention relates to a process involving filtrates, more particularly an improvement of the process which extends the significance of filtrate and its multilevel multi-components.

### DESCRIPTION OF RELATED ART

Filtrate extraction is explored by many scientific methods and is available in large numbers. But filtrate extension has not reflected the public in any reference documents. Filtrate extraction is publicly worked invention whereas filtrate extension is based on the principle of combination theory that single filtrate does not have significant. superiority as compared to the combination of filtrates. Rigorous demonstration of combination effect involves a lot of science with inherent results in the product, steps for the desired function, method of changing functionalities, workshop improvements and due diligence exercise is not conducted by any synthetic chemist, API manufacturers, polymer science and so on. Filtrate synergism is unencountered in any technology. Filtrate extension in an organic media comprising polar protic and polar aprotic solvents by suitable means is purely technical as compared to existing knowledge and of economic significance. Filtrate extension and filtrate synergism is not anticipated by the public and does not form part of the state of the art is one of the substrates as evidenced in VSN14, VSN16, and VSN19.

The traditional multicomponent reaction is carried out in one pot in multi steps leads to drug product. Large number of GLP, GMP, and API manufacturers rely upon convergent and divergent reaction strategy. Any stereogenic centre's drugs can be obtained as lead product but other isomer or byproducts is escaped out in the filtrate as an unrecovered product. The aforesaid utility model is provided a useful addition to the stock of human knowledge which is the reason for the existence of patents. The knowledge is on filtrate, for filtrate, through filtrate and hence filtrates to filtrate and its multicomponent and multilevel process is needed to be improved.

Multicomponent reactions MCR'S are widely acclaimed in reactions like Ugi's reaction, Suzuki coupling, and such reactions are categorized as tri, tetra, penta, components and so on. The components and levels are retained here in a convergent, divergent reaction strategy but change adopted as auxiliary organic media.

The motto behind this term is that“like solvents dissolve likes”, the substrates are dissolving in one another forming homogenous mixture which is conducive for chemical transformations.

The said concept is not claiming multicomponent and multilevel but the method by which substrate fragments infiltration is illustrated in auxiliary bi organic to deca organic media. The outcome of the sequential experiments leads to small to polycarbon containing compounds with benevolent properties.

### SUMMARY OF THE INVENTION

The main object of the present invention is an improvement to rationalize substrates in the filtrate by condensation rather than the methodology of MCR depending on acidic or basic conditions of filtrates at a higher level aimed to synthesize and showcase the diverse assemblies of medicinally important small molecular libraries of great concern.

The other object of the present invention is extending Filtrate to filtrate extension asserted in ornamenting of substrate to produce chemical auxiliaries distinctive in its pharmaceuticals.

In other words our objective is to generate filtrate libraries in anon obvious manner.

*yet other objective of the present invention is to develop chemical auxiliaries with improved stability, solubility, lipophilicity is the statutory requirement of utility, novelty in the formation The other objective of the present invention is to expand from a low molecular weight compound (scouting library) to large library with the development physiochemical tools and increase the yields often possible from preclinical lab scale (mg, gms) to clinical amounts (kgs) using said technology.

Another objective is to improve the process to give advanced compounds with high-density atoms thereby filtrate extension technology manifests drug discovery chemistry. A multitude of filtrates is useful in defining chemical space can be ensemble in the synthesis. Attempt to qualify green credential is still ongoing research and matrices of this technology may quantify greener chemical reactions. This technology is not compelled to use hazardous materials and process does not require heating so that the idea of renewability of the materials used is maintained at different levels.

### DETAILED DESCRIPTION OF THE DRAWINGS

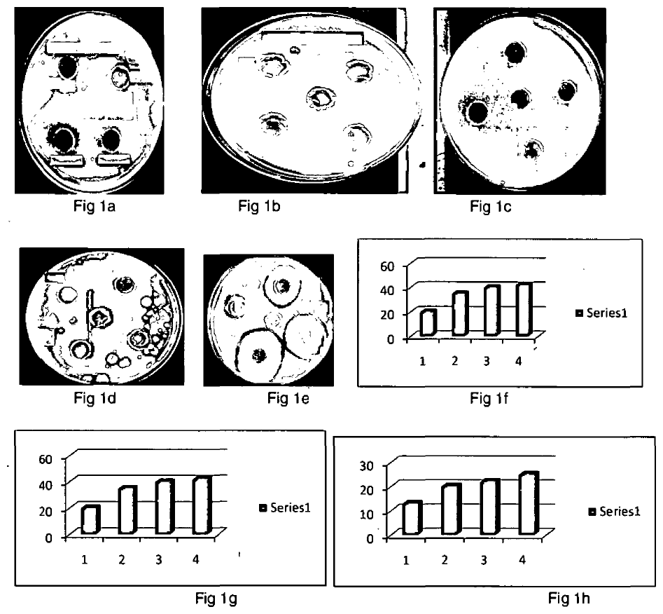

The FIG. 1a-li attributes to compound 2R derived from filtrate library of picric acid, sodium azide, chloroacetic acid, hydrazine hydrate, benzaldehyde mixture suitably mixed with salicyaldehyde, acetamide, formaldehyde mixture so that composition 2R has remarkable biological activities.

Fgl a-FIG. 1 illustrates that the present invention uses a modified filtrate extension process helps Antimicrobial, Antifungal, Antibacterial assay results from table and graph to show that the test sample is having good anti-fungal activity. The bioactivity is also found to be concentration dependent activity increased with an increase in the amount of test sample

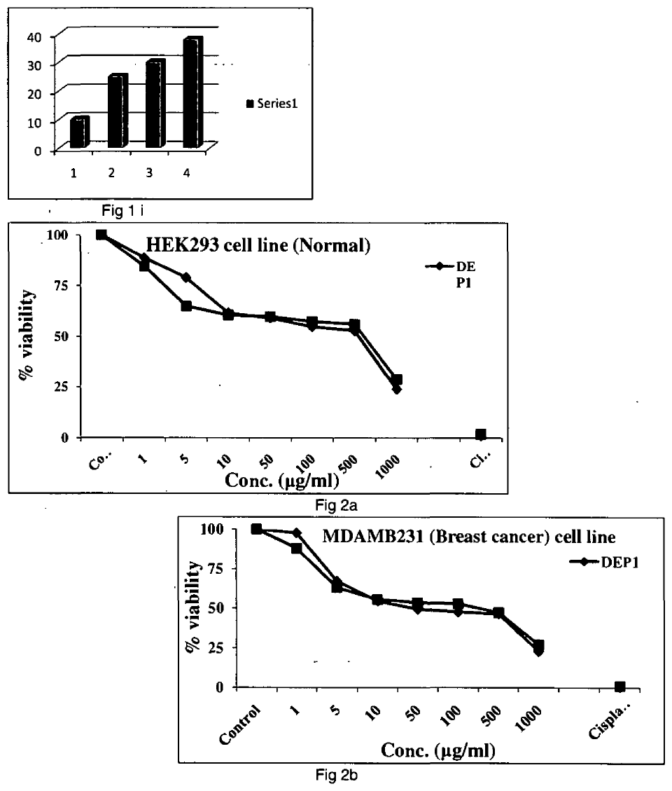

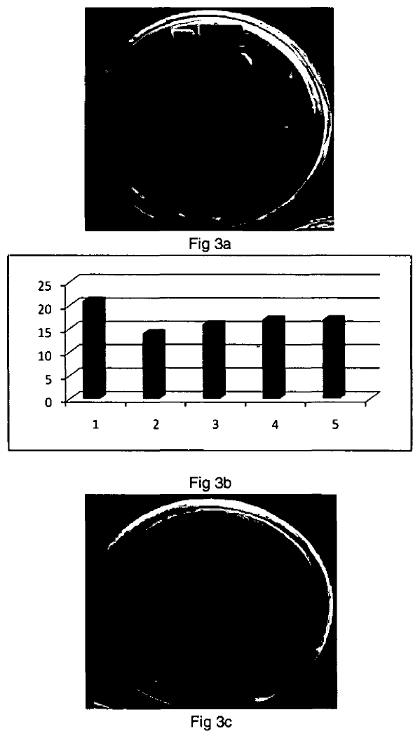

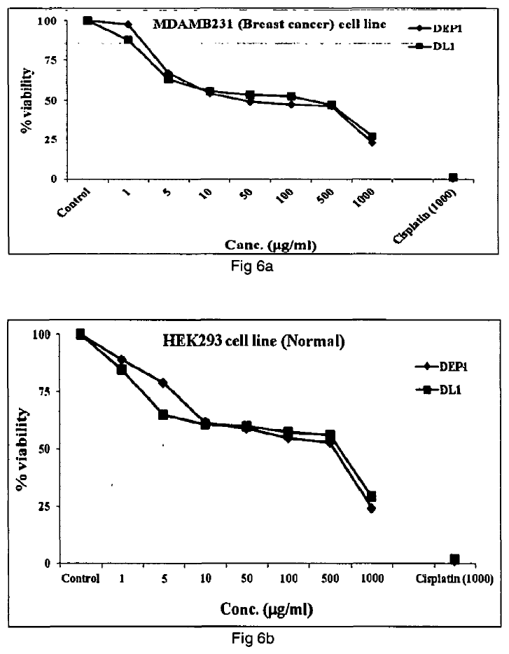

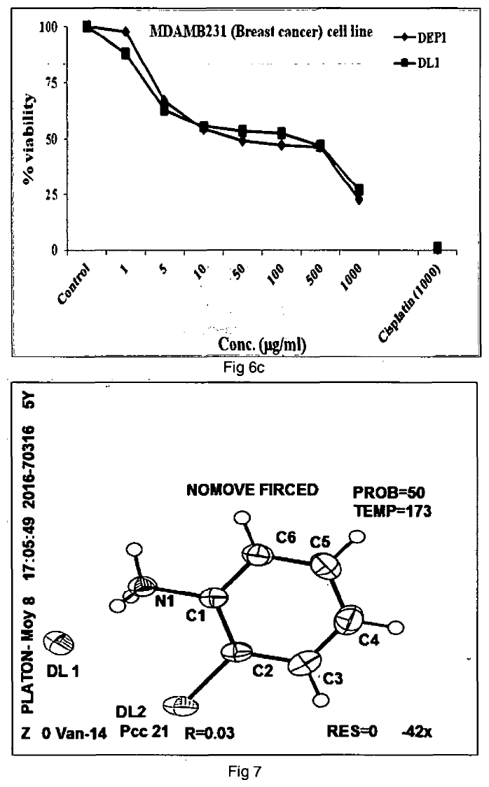

The commercial antiseptic solution containing ethanolamine, amyl alcohol, phthalic anhydride mixtures in phosphoric acid resulted in DEP1 while orthophenylenediamine, ethyl acetoacetate under suitable condition forming yellow precipitate DL1 whose properties are summarized in the Filtrate extension of drugs DEP1 and DL1 whose concentration dependence are evident in impedance curve. FIG. 6a-6c FIG. 2a, 2b illustrate the graph explaining the improvement of the process ethanolamine, amyl alcohol, phthalic anhydride, in commercial antiseptics, resulted DEP1, showed cytotoxic, anticancer behavior and MCR reactant orthophenylenediamine, ethyl acetoacetate, phenylacetic acid in commercial antiseptic mixture resulted DL1, showed the anticancer property.

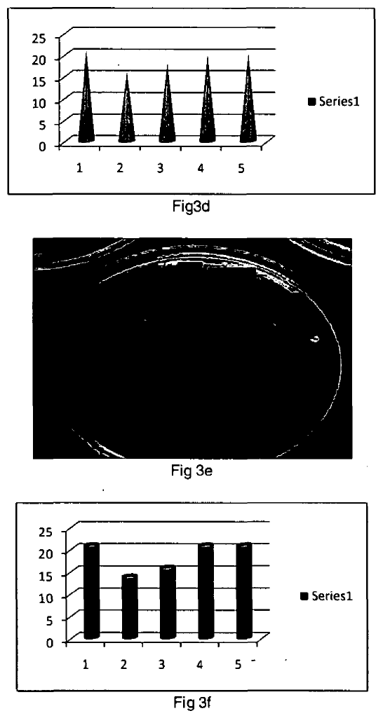

The compound activities of K resulted by filtrate extension of para-aminobenzoic acid, para-toluene sulphonamide followed by activation and condensation with sodium azide giving yellow solid whose clinical importance is depicted in the drawings. {FIG. 3a-3f)

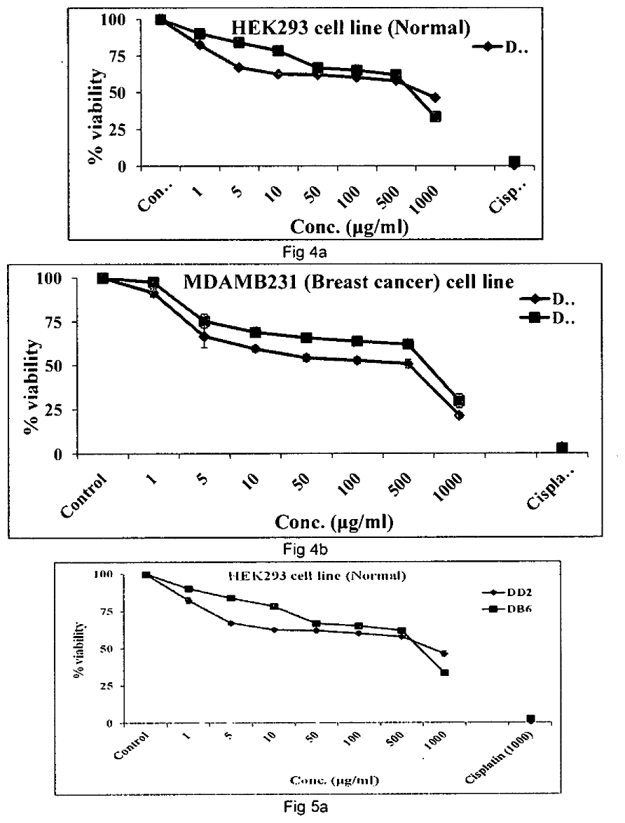

FIG. 3a-3f illustrate Antibacterial activity of K compound using Zone of Inhibition method and Anti-Yeast activity of K compound using Zone of Inhibition Method Drugs DEP1 and DL1 are prepared by filtrate synergism mentioned in the report whose concentration dependence is evident in the impedance curve. (FIG. 4a-6c)

FIG. 4a, 4b illustrate the graph showing HEK293CELL LINE AND MDA-MB 231 (breast cancer) cell line where it explains MDA-MB 231 is basal, aggressive cell line of higher growth rate, metastatic ability which has unique growth pattern but on treatment of drugs at different concentration and dose-dependent decrease in cells viability and percentage of viability at 1000 microgm/dl is seen in the impedance curve

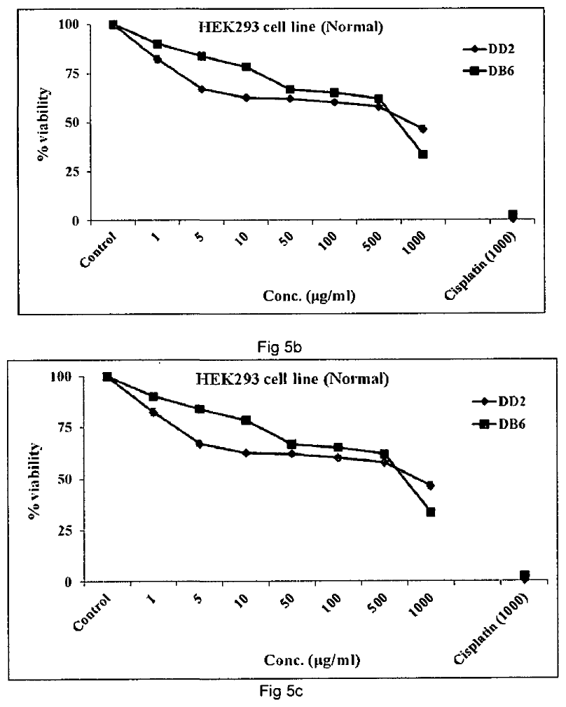

FIG. 5a-5c illustrate the reaction The MTT (3-(4, 5-dimethylthiazol-2-yl)-2, 5-diphenyltetrazolium bromide) assay as a simple colorimetric assay for screening cell viability, depends on cellular NAD(P)H oxidoreductase enzymes of live cells and in the graph percentage viability of HEK293 cell line with respect to different concentrations of DD2 and DB6 samples, the percentage viability of MDA-MB 231 cell line with respect to different concentrations of DD2 and DB6 samples.

FIG. 6a-6c illustrate the reaction where MTT (3-(4, 5-dimethylthiazol-2-yl)-2, 5-diphenyltetrazolium bromide) assay is a simple colorimetric assay for screening cell viability, depends on cellular NAD(P)H oxidoreductase enzymes of live cells. The mitochondrial succinate dehydrogenase from live cells which reduces yellow 3-(4, 5-dimethythiazol2-yl)-2, 5-diphenyl tetrazolium bromide (MTT) to an insoluble, dark purple colored formaz an crystals and graphs percentage viability of HEK293 cell line with respect to different concentrations of DEP1 and DL1 samples, The percentage viability of MDA B231 cell line with respect to different concentrations of DEP1 and DL1 samples

FIG. 7 illustrate filtrate performance by the alternative process is possible through bilayer filtrate of orthochloroaniline, hydroxylamine hydrochloride in the scouting library molecule orthochloroaniliniumhydrochloride which is crystallized in the filtrate.

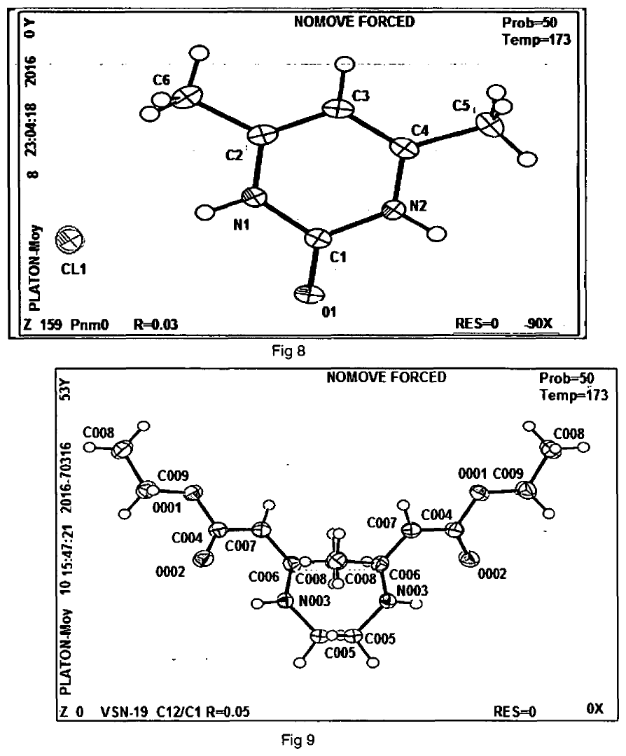

FIG. 8 illustrate the formation of pyrimidine derivative in a facile way through filtrate of ethylacetoacetate, urea, under suitable condition by condensation only but not covered in the prior art.

FIG. 9 illustrates an example for polycarbon contains a compound of filtrate synergism ethyl acetate, ammonium carbonate, acetyl acetone by suitable means whereby chelate formation, complexation reactions in the biological, pharmaceutical preparation is possible in a cost-effective manner.

For a-10b attributes to TZ2 and TZR whose absorbance is more valued rather than image since light rays bring the destruction of cellular activities opinioned by research head at Udupi health science research center.

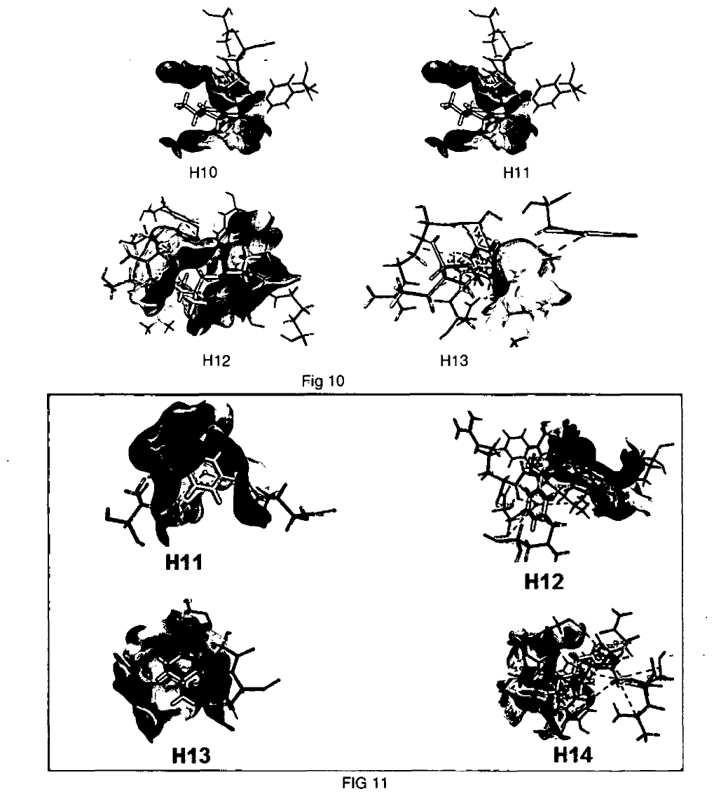

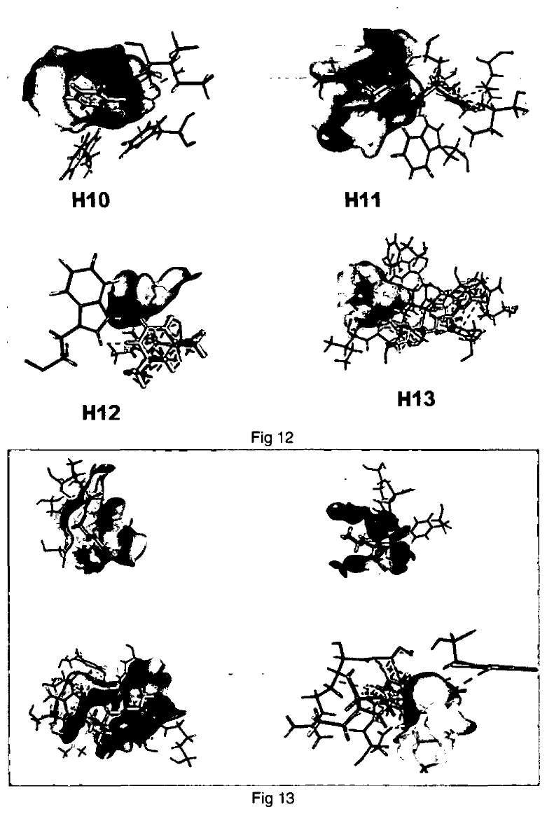

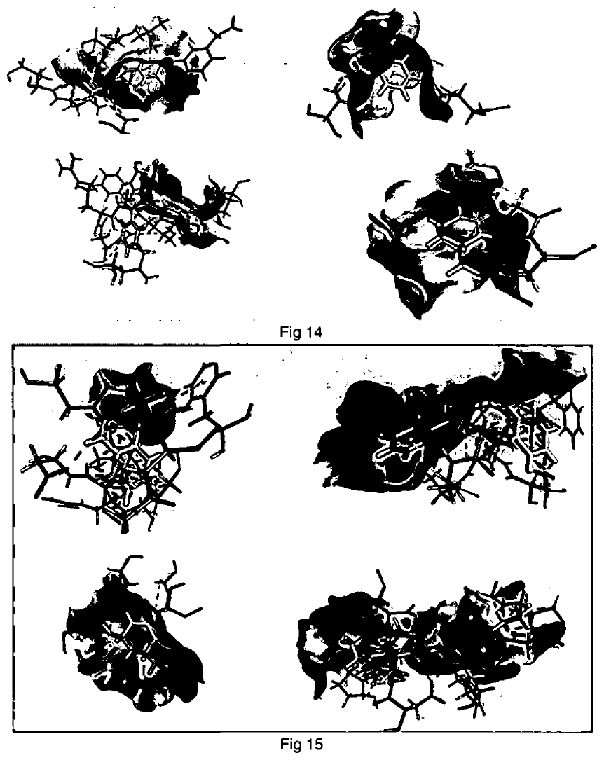

FIG. 10a and FIG. 10b9 (introduced graph) illustrate an example of graph showing anticancer activity of HEK293CELL LINE AND MCF-7 breast cancer cell line Less aggressive, non invasive, relatively slow growing, hormone-dependent cell line also capable of reacting with filtrate derived products TZ2, TZR by the multicomponent of thiazolidinone, picric acid, hydrazine hydrate under suitable conditions of acid/base treatments showed positive to real-time impedance analysis on the said cell line.

Table 9 and 10 showed the percentage of viability values of the samples TZ2, TZR at different concentrations MCF-7 is although relatively resistant to cisplatin treatment compared to other breast cancer cell lines, the drug dependent curve indicates antiproliferative to cytotoxic efficacy value which is useful for the study of cancer progression.

| | Anti- | Anti- | | Anti- | Anti- |
|---|---|---|---|---|---|
| compound | bacterial | fungal | Anthelmintic | inflammatory | cancer |
| 10 | −0.847 | −0.836 | −3.552 | −13.263 | −13.337 |
| 11 | 1.015 | −3.307 | −4.886 | −8.468 | −12.101 |
| 12 | 2.681 | −0.938 | −4.267 | −13.364 | −11.855 |
| 13 | −3.885 | −3.307 | −3.615 | −13.073 | −13.176 |

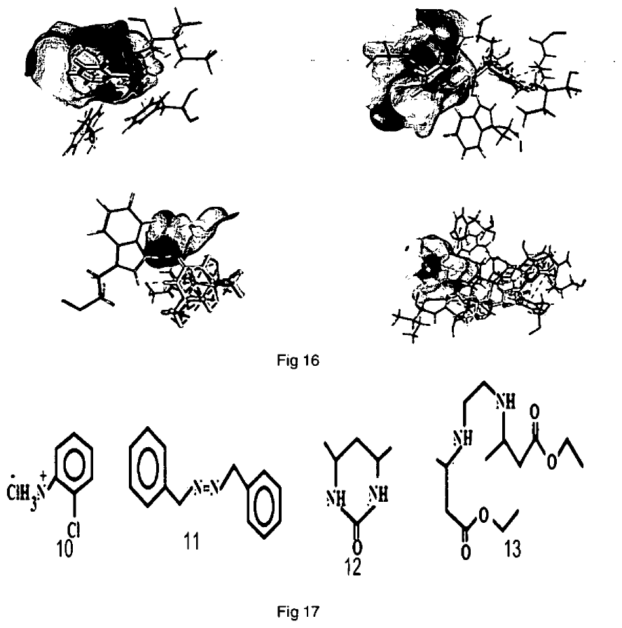

FIG. 10 to-FIG. 17 illustrate the docking summary of compounds 10 compound 11, compound 12 compound 13 depicted in the columns]

### DETAILED DESCRIPTION OF THE INVENTION

Filtrates of organic reactions in the organic media (polar protic/polar aprotic)contain molecular fragments are obvious and method to get chemical auxiliaries is not obvious to a person skilled in the art.

Organic reaction by convergent or divergent approach is most common for a synthetic chemist, researchers. New approach in which EHS (Environment, Health, Safety) reducing use of energy, low business risk are precedence in the technology. The filtrates of multi components at multilevel is modulated under suitable conditions and the steps per se is unobvious. The claim for process of manufacture involves key elements of pH condition, less hazardous chemicals, economy of solvent and cost effective combinations lead to novel molecular diversity by alternative methods

Where Filtrate extension process is derived from bi organic to deca organic reaction media so that scouting libraries to complex chemical auxiliaries are formed at low cost

The MCR filtrates are purported in an organic media then alleged by combining filtrate of another multicomponent reaction become an effective organic media. This is created by changing pH, acid/base treatment, substrate combination so that new chemical auxiliaries result under normal laboratory condition. One can reduce the bulk by concentration if necessary.

Hereby the Filter off the precipitated chemical auxiliary (if needed) and same filtrate were carried forward to next level for the third set of multicomponent reaction, or by adding new substance to the mixture and Saturate the filtrates by concentration and allowed to stand under laboratory condition.

The main object in the present invention is to improve the process to increase the performance of a reaction by filtration extension where the improvement in the process comprising filtrate 1 and filtrate 2 from the reactions of mixture 1 and mixture 2 respectively, and mixing the filtrate 1 and filtrate 2 in the next reaction, wherein the reaction condition is controlled by controlling pH of the mixture 1 and mixture 2, acid/base treatment, resulting plurality of filtrate extension chemical auxiliaries in higher level of organic media

If the acid used in the treatment step is concentrated sulphuric acid and base is potassium hydroxide pellets, All the inventive steps for filtrate is carried out in a vessel may consist essentially of an untreated substance; broken molecular fragments are ornamented in the new product. The level of ordinary skill in the pertinent art and unsolved needs of filtrate extension, synergism, can tell it to people how it can be usefully employed

The present invention relates to technical aspects, such as inventive step and expected effect is on various industries.

### Example 1

The reaction treatment step in using conc. H2S04 and KOH pallets at the right time, the right situation, change in pH, use of substrate attributes in respect to its purpose of use which is not previously known.

The product defined by the process as in VSN14, VSN16, VSN19 is solely formed by this technology, which is not available/not covered in the prior art

The publically worked invention of VSN14, VSN16, VSN19 is eighter by oxidation, condensation, reduction, as a major process but by the claimed invention, has the inherent feature of filtrate synergism. There is a difference between prior technology and actual invention in all the aforesaid products.

The practical applicability of the invention is mentioned in compound K, 2R, DEP, DL1, EG4S, DB6, TZ2, and TZR

### Example 2

Standard Zone of inhibition Assay was carried out to test anti-microbial activity.

The test sample was dissolved in water to make 10% solution. The different concentrations of test sample 25 microliter, 50 microliters, 75 microliters, and 100 microliters was used for activity assay.

Antibacterial assay results from the above table and graph show that the test sample is having good anti-bacterial activity. The bioactivity is also found to be concentration dependent, activity increased with an increase in the amount of test sample.

Where Antibacterial assay results from the above table and graph show that the test sample is having good anti-fungal activity. The bioactivity is also found to be concentration dependent activity increased with an increase in the amount of test sample.

TABLE 1
Activity parameters

| Dosage in miclt | Bacteria(zone in MMS) |
|---|---|
| 25 | 20 |
| 50 | 35 |
| 75 | 40 |
| 100 | 42 |

TABLE 2
Activity parameters

| Dosage in miclt | Fungus(zone in mms) |
|---|---|
| 25 | 13 |
| 50 | 20 |
| 75 | 22 |
| 100 | 25 |

TABLE 3

| Dosage in miclt | Yeast (zone in mms) |
|---|---|
| 25 | 10 |
| 50 | 25 |
| 75 | 30 |
| 100 | 38 |

Antibacterial assay results from the above table and graph show that the test sample is having good anti-fungal activity. The bioactivity is also found to be concentration dependent activity increased with an increase in the amount of test sample.

The test sample shows broad spectrum anti-microbial activity. The anti-yeast activity is the highest and long lasting showing inhibition even after two weeks.

Filtrate extraction & filtrate extension are 2 separate domains and the scope of creating organic media connotes filtrate extension.

This technology is adoptable to all convergent and divergent reaction without recourse to chemical hazards, toxic chemicals, but cost effective manner even to prepare small library to complex library.

In the embodiment of the invention the VSN14, VSN16, VSN19 in a substantially pure form from auxiliary organic media used.

The other embodiment the method, reactant comprises are p-toluene sulphonamide, para amino benzoic acid, reactants filtrates in glacial acetic acid connotes to compound K showed antimicrobial, antifungal, anti-yeast properties.

Table 4

The other embodiment, the reactants comprises of picric acid, benzaldehyde, salicylaldehyde, acetamide, formaldehyde, construed in 2R showed the antimicrobial property.

In other embodiment the filtrate multicomponent reactants comprise ethanolamine, amyl alcohol, phthalic anhydride, in commercial antiseptics, resulted in DEP1, showed cytotoxic, anticancer behavior and reactant comprise orthophenylenediamine, ethyl acetoacetate, phenylacetic acid in commercial antiseptic mixture resulted DL1, showed the anticancer property

wherein the reactants comprise ethylenediamine, glycerol, thiophene 2 carboxylic acid in a commercial antiseptics, resulted in EG4S showed antibacterial, antimicrobial assay test. And the reactants comprises benzyl chloride, ethanolamine thiophene-2-carboxylic acid, in phosphoric acid resulted DBS showed positive towards MTT assay.

### Example 3

Cytotoxic and Anticancer screening by MTT assay.

Selection of Cell line and preparation of medium:

The cytotoxicity and anticancer screening were carried out using Human Embryonic Kidney (HEK293) and MDAMB231 (breast carcinoma) cells lines respectively. The cell lines were procured from National Centre for Cell Science (CCS), Pune and cells were grown using Minimum Essential Medium Eagle (MEM) and Leibovitz-15 (L-15) with 10% fetal bovine serum as per standard respectively.

Principle:

The MTT (3-(4, 5-dimethylthiazol-2-yl)-2, 5-diphenyltetrazolium bromide) assay is a simple colorimetric assay for screening cell viability, as per the standard procedure.
Calculated the percentage of viable cells using the following formula:

% of viable cells=[(Test sample−blank)/(Control−blank)]×100

Results:

TABLE 1
The percentage viability of HEK293 cell line with respect
to different concentrations of DD2 and DB6 samples. Table 5

| | |
|---|---|
| | % viability |
| HEK293 cell line | Mean value |

| (μg/ml) | DD₂ | DB₆ |
|---|---|---|
| Control | 100 | 100 |
| 1 | 82.591 | 90.269 |
| 5 | 67.216 | 84.138 |
| 10 | 62.649 | 78.549 |
| 50 | 62.179 | 66.921 |
| 100 | 60.228 | 65.268 |
| 500 | 57.902 | 62.022 |
| 1000 | 46.474 | 33.499 |
| Cisplatin (1000) | 0.476 | 2.737 |

TABLE 6

| | |
|---|---|
| | % viability |
| MDAMB231 cell line | Mean viability |

| (μg/ml) | DD₂ | DB₆ |
|---|---|---|
| Control | 100 | 100 |
| 1 | 91.643 | 97.540 |
| 5 | 66.663 | 75.431 |
| 10 | 59.375 | 68.895 |
| 50 | 54.255 | 65.776 |
| 100 | 52.787 | 63.702 |
| 500 | 50.903 | 61.997 |
| 1000 | 21.433 | 29.741 |
| Cisplatin (1000) | 3.392 | 2.619 |

Conclusion:

At different concentrations of DD2 and DB6 showed dose-dependent decrease in the cell viability with respect to increase in the concentration of samples used in the HEK293 cell line. The drug uBb showed less cytotoxicity effect from 1-500 pg/ml whereas it showed more cytotoxicity effect at 1000 pg/ml. Similarly, the drug DD2 showed more cytotoxicity effect from 1-500 pg/ml, whereas it showed comparatively lesser cytotoxicity effect at 1000 pg/ml concentration. The positive control Cisplatin at 1000 (pg/ml) showed 0.476% and 2.737% of cell viability.

The different concentrations of DD2 and DB6 showed a dose-dependent decrease in the cell viability with respect to an increase in the concentration of samples in the MDAMB231 cell line. DD2 showed more anticancer property compared to DB6 with respect to different concentrations of samples. The positive control Cisplatin at 1000 (pg/ml) showed 3.392% and 2.619% of cell viability.

### Example 4

Cytotoxic and Anticancer screening by MTT assay.

Selection of Cell line and preparation of medium:

The cytotoxicity and anticancer screening were carried out using Human Embryonic Kidney (HEK293) and MDAMB231 (breast carcinoma) cells lines respectively. The cell lines were procured from National Centre for Cell Science (NCCS), Pune and cells were grown using Minimum Essential Medium Eagle (MEM) and Leibovitz-15 (L-15) with 10% fetal bovine serum as per standard respectively.
Calculate the percentage of viable cells using the following formula:

% of viable cells=[(Test sample−blank)/(Control−blank)]×100

Results:

TABLE 7
The percentage viability of HEK293 cell line with respect
to different concentrations of DEPi and DLi samples.

| | |
|---|---|
| | % viability |
| HEK293 cell line | Mean value |

| (μg/ml) | DEPᵢ | DLᵢ |
|---|---|---|
| Control | 100 | 100 |
| 1 | 88.765 | 84.486 |
| 5 | 78.815 | 64.883 |
| 10 | 61.454 | 60.451 |
| 50 | 58.913 | 59.686 |
| 100 | 54.654 | 57.284 |
| 500 | 52.741 | 55.950 |
| 1000 | 23.854 | 28.853 |
| Cisplatin (1000) | 0.958 | 1.801 |

TABLE 8

| | |
|---|---|
| | % viability |
| MDAMB231 cell line | Mean value |

| (μg/ml) | DEP1 | DL1 |
|---|---|---|
| Control | 100 | 100 |
| 1 | 97.594 | 87.956 |
| 5 | 67.218 | 63.194 |
| 10 | 54.370 | 55.504 |
| 50 | 49.249 | 53.401 |
| 100 | 47.495 | 52.646 |
| 500 | 46.351 | 47.111 |
| 1000 | 22.884 | 26.999 |
| Cisplatin (1000) | 0.483 | 1.070 |

Conclusion:

At different concentrations of DEP-i and DLi showed dose-dependent decrease in the cell viability with respect to increase in the concentration of samples used in the HEK293 cell line. Overall DEPi and DLi did not show much difference in cytotoxicity effect between 1-1000 pg/ml. The positive control Cisplatin at 1000 (pg/ml) showed nearly 0.958% and 1.801% of cell viability.

The different concentrations of DEPi and DLi showed dose-dependent decrease in the cell viability with respect to increase in the concentration of samples in the MDAMB231 cell line. At lower concentration of DEPi (1-10 pg/ml) showed less anticancer activity and at higher concentration (10-1000 pg/ml) DEPi showed comparatively more anticancer effect compared to DLi. The positive control Cisplatin at 1000 (pg/ml) showed nearly 0.483% and 1.070% of cell viability.

TABLE 9

| | | | Mean | SD | SE | | | | |
|---|---|---|---|---|---|---|---|---|---|
| HEK293 | Control | | 100 | 0 | 0 | | | TZR | TZ2 |
| TZR | 10 | μg | 40.458 | 2.873 | 2.032 | Control | | 100 | 100 |
| | 20 | μg | 35.668 | 2.982 | 2.109 | 10 | μg | 40.458 | 22.771 |
| | 50 | μg | 27.545 | 0.585 | 0.414 | 20 | μg | 35.688 | 17.076 |
| | 100 | μg | 9.242 | 0.631 | 0.446 | 50 | μg | 27.545 | 13.466 |
| | 200 | μg | 7.210 | 1.298 | 0.918 | 100 | μg | 9.242 | 9.153 |
| | 500 | μg | 4.411 | 0.077 | 0.055 | 200 | μg | 7.210 | 8.157 |
| | Cisplatin | | 2.490 | 0.211 | 0.149 | 500 | μg | 4.411 | 5.369 |
| | (500 μg) | | | | | | | | |
| | | | | | | Cisplatin | | 2.490 | 1.929 |
| | | | | | | (500 μg) | | | |
| | | | Mean | SD | SE | | | | |
| HEK293 | Control | | 100 | 0 | 0 | | | | |
| TZ2 | 10 | μg | 22.771 | 4.170 | 2.949 | | | | |
| | 20 | μg | 17.076 | 3.340 | 2.362 | | | | |
| | 50 | μg | 13.466 | 1.318 | 0.932 | | | | |
| | 100 | μg | 9.163 | 1.109 | 0.784 | | | | |
| | 200 | μg | 8.157 | 1.421 | 1.005 | | | | |
| | 500 | μg | 5.369 | 0.367 | 0.260 | | | | |

| | | | |
|---|---|---|---|
| Cisplatin | 1.929 | 0.512 | 0.362 |
| (500 μg) | | | |

TABLE 10

| | | | Mean | SD | SE | | | | |
|---|---|---|---|---|---|---|---|---|---|
| MCF-7 | Control | | 100 | 0 | 0 | | | TZR | TZ2 |
| TZR | 10 | μg | 45.784 | 2.488 | 1.759 | Control | | 100 | 100 |
| | 20 | μg | 38.881 | 3.732 | 2.639 | 10 | μg | 45.784 | 37.832 |
| | 50 | μg | 35.653 | 2.256 | 1.595 | 20 | μg | 38.681 | 30.093 |
| | 100 | μg | 34.014 | 0.339 | 0.240 | 50 | μg | 35.653 | 29.431 |
| | 200 | μg | 28.394 | 0.875 | 0.619 | 100 | μg | 34.014 | 23.388 |
| | 500 | μg | 24.985 | 0.931 | 0.658 | 200 | μg | 28.394 | 21.287 |
| | | | | | | 500 | μg | 24.985 | 17.817 |
| | Cisplatin | | 11.851 | 1.010 | 0.714 | | | | |
| | (500 μg) | | | | | | | | |
| | | | | | | Cisplatin | | 11.851 | 13.263 |
| | | | | | | (500 μg) | | | |
| | | | Mean | SD | SE | | | | |
| MCF-7 | Control | | 100 | 0 | 0 | | | | |
| TZ2 | 10 | μg | 37.832 | 2.221 | 1.571 | | | | |
| | 20 | μg | 30.093 | 2.437 | 1.723 | | | | |
| | 50 | μg | 29.431 | 2.165 | 1.531 | | | | |
| | 100 | μg | 23.388 | 1.191 | 0.842 | | | | |
| | 200 | μg | 21.287 | 0.876 | 0.620 | | | | |
| | 500 | μg | 17.817 | 0.563 | 0.391 | | | | |

| | | | |
|---|---|---|---|
| Cisplatin | 13.263 | 0.689 | 0.487 |
| (500 μg) | | | |

The protocol HEK293 AND MCF-7 cell lines were procured from National center for cell science (NCCS), Pune and cells are grown using MEM (E) with NEAA and 10% Fetal Bovine Serum(FBS) as per standard instruction.

Conclusion:

The differential concentration of coded TZ2, TZR drugs showed dose-dependent cell proliferation by decreasing cell viability by the loss of ability to reduce tetrazolium products. At the end of exposure period level of metabolically active cells are abnormally reduced and higher cytotoxic and anticancer properties with respect to HEK293, and MCF-7 cell lines are evidenced at the experimental conditions.

In other embodiment of the invention in any reaction with filtrate extension medium results significant improvement on their organic level where reaction pathway observed is fragment filtrate extension, The products derived from. Auxiliary, organic media is of qualitative as well as quantitative.

Filtrate of reaction bound to contain molecular fragments is suitably converted at normal conditions.

### Example 5

Mixture of ortho-ch!oroaniline and meta-nitro benzoic acid in one reaction setup form white precipitate 9 and extension of filtrates with flavanoids such as flavanone, quercetin, diosmin, & rutin become auxiliary triorganic in acidic condition form white solid to plate like solids (AI F.AIQ.AI D.ATR)

### Example 6

Thiophene 2-carboxylic acid in ammonical hydrazine followed by nitrosation white precipitate TD is formed and extended reaction in filtrate media resulting yellow powder T3 with o-methoxybenzaldehyde brown needle T5a with cinnamic acid, colourless needles are formed with phenyl acetic acid T7, plate like crystals with benzoic acid T8, & reaction condition become auxiliary triorganic.

Example 7 Hydroxylamine hydrochloride, nitrous acid reaction condition benzaldehyde and phenyl hydrazine combine to give yellow ppt D1, while benzaldehyde& 0-Hydroxyacetophenone resulting yellow ppt TF1, bromine in alkaline condition both benzaldehyde& 0-hydroxyacetophenone form green precipitate F-n

Salicylic acid and orthophenylenediamine in successive steps aci/base contribution resulting white precipitate SP which is (A20M)

### Example 8

P-amino benzoic acid and p. Toluene sulphonamide by activation followed by sodium azide in acetic acid media give bioactive K2 is nothing but (A20M)

### Example 9

Formation of the products between o-chloroaniline, m-Nitrobenzoic acid and flavanone conclusively A30M

### Example 10

Thiophene 2-carboxylic acid in successive steps form T3, T5a, T7, T8, TS is A30M.

### Example 11

Mixture of acetamide, p-nitro aniline, benzaldehyde or its derivative can be suitably treated to give bioactive M2

### Example 12

O-phenylenediamine and ethyl acetoacetate in presence of meta nitrobenzoic acid under acid/base treatment form yellow precipitate E2.

### Example 13

Mixture of o-hydroxyacetophenone, formic acid, resorcinol in successive steps give white ppt IF12

Example 14 Salicyaldehye, acetamide& Formaldehyde under suitable condition form white ppt RU is found to be bioactive.

### Example 15

Nitrosation of aspirin, ethanolamine.ochloroaniline and phenyl hydrazine in acidic media give whitish powder AC which is auxiliary tetra organic media product (A40M)

### Example 16

Using glycerol as solvent compound, p-cresol, benzanilide.Flavanone and hippuric acid under acidic media resulted yellow solid P3

### Example 17

In the formic acid media anthranilicacid, benzyl chloride, phenyl hydrazine and flavanoids form compound series A-i, BI, C1, D2 but in hydrazine acetic acid form A2, B2, C2, and D2 product.

### Example 18

Picric acid is condensed with chloroaceticacid, bromosuccinamide in presence of phenyl hydrazine give yellowish white precipitate (8) and filtrate extension in o-chlorobenzaldehyde and hydrazine converted to yellow powder 2CBA which is (A50M)

### Example 19

m-nitrobenzoic acid, urea, phenyl hydrazine in acetonitrile solvent form buff ppt B3C3 is another example of A40M.

### Example 20

In acetonitrile solvent p-nitroaniline, acetamide, benzaldehyde condense to form white precipitate M4, but in ethanolamine yellow precipitate M5

### Example 21

Nitrosation reaction between hippuric acid, ethanolamine, p-Toluene sulphonamide and sulphanilic acid form greenish ppt N3

### Example 22

In glycerol solvent nicotinic acid, flavanone, ethnolamine condense to give brown take EG2 instead of benzanilide same mixture with nicotinic acid form yellow powder EG3. Under ethanolamine & glycerol thiophene 2-carboxylic acid & diosmin give brownish white solid EG4 which is an example of A40M.)

### Example 23

Auxiliary compound obtained mixing five organic compound in suitable condition form derivatives is nothing but (A50 M). Some of the examples are.

i) Mixture containing benzyl chloride, p-dibromobenzene, benzanilide, quercet in ethanolamine form brownish needle B2.

### Example 24

Above mixture instead of quercetin is replaced by flavanone yellow ppt B3 is formed

### Example 25

Replacing benzanilide, by nicotinic acid above reaction condition yellow crystaline solid B5 is resulted.

### Example 26

Replacing benzanilide & quercetin in mix.1. by thiophene 2 carboxylic acid and diosmin above condition give brown needle B6.

### Example 27

Mixture containing benzinilide, p-dibromobenzene phenyl hydrazine, ortho phenylene diamine in ethyl acetoacetate in acid base condition give brown solid BDE is another example of A50M.

### Example 28

Combination of aniline or its derivatives, formic acid, urea in triethanol amine solvent condenses with chloroacetic acid give pinkish white solid PU.

### Example 29

Pentaorganic containing salicyaldehyde, benzamide, formaldehyde & phenyl hydrazine condenses with urea giving yellow precipitate RHR.

### Example 30

In mix no. 7 is replaced phenyl hydrazine is replaced o-hydroxyacetophenone and urea is replaced by benzaldehyde white solid RF11 is obtained.

### Example 31

Hexa organic media contain benzylchloride, benzamide, salicylic acid.

Formaldehyde StThiophene 2-carboxylic acid in Ethanolamine solvent resulted white ppt ERD.

### Example 32

Mixture containing hippuric acid, Flavanone, benzanilide, p dibromobenzene& Formic acid in alkaline media give yellow ppt DAE.

### Example 33

Mix no. 2 with quercetin under same condition give yellow ppt D2BE.

### Example 35

Mix no. 2 with Diosmin in alkaline condition gives white ppt D3BE.

### Example 36

Hexaorganic containing benzamide, phenyl hydrazine, ascorbic acid, acetamide, 0 phenylenediamine in ethanolamine solvent allowed to stand for few hours yellow ppt HP is formed.

### Example 37

Mix no. 5 instead of o-phenylenediamine, thiophene 2-carboxylic acid is placed in the RB flask stirred for 6 hrs give white precipitate H5

### Example 38

Another Hexa organics containing benzyl chloride, p-dibromobenzene, pToluidine, nicotinic acid, quercetin in ethanol amine give yellow-needle\hat{ }on allowed to stand Biorganic filterate A2 is coupled with m-nitrobenzoic acid, chloroacetic acid, sulphanilic acid and N bromosuccinamide precipitate DA2

### Example 39

A flavanoid quercetin reacts with mixture containing benzanilide, m-nitrobenzoic acid, chloroacetic acid, sulphanilic acid, N-bromosuccinamide, ethyl acetoacetate solvent give canary yellow precipitate QB

### Example 40

Hepta organic mixtures containing m-nitrobenzoic acid, chloroacetic acid, sulphanilic acid, n-bromosuccinamide, benzanilide and flavanone in ethyl acetoacetate form pinkish white ppt FB4.

### Example 41

Instead of benzanilide, &flavanone treated with nicotinic acid & pToluidine to the mixture no. 3 form yellow precipitate QB3.

### Example 42

Mixture containing picric acid, chloroacetic acid, N-bromosuccinamide, benzaldehyde, salicylic acid, 2,4 dihydroxyacetophenone and acetophenone in ammonical media form yellow solid SA

### Example 43

Mixture of p-bromobenzene, sulphanilic acid, urea, acetyl acetone, ethanol amine, o chlorobenzalhyde in acetic acid shacked for 4 hrs to give Yellow powder CJ.

### Example 44

Mix no. 5 of hexaorganic media condense with salicylic acid in ethyl acetoacetate solvent give yellow ppt. HER.

### Example 45

Another hepta organic mixture containing benzanilide, Diosmin.benzaldehyde, accetophenone& Urea, p Toluidine in ethylene diamine Solvent give yellow precipitate PH3.

### Example 46

Mixture of aliphatic & aromatic aldehyde condense with acetamide&benzamide in presence of salicylic acid p nitroaniline in ethyl acetoacetate solvent yellow ppt RM5 Mixture of eight different organic substrate under suitable reagent Converted to auxiliary compound is (A80M).

### Example 47

Combination of component containing benzanilide, p-dibromobenzene, phenyl hydrazine & Formic acid is agitated with another components containing m-nitrobenzoic acid, chloroacetic acid, p-Toluene sulphonamide in ethyl acetoacetate give yellow crystalline solid (BDOA

### Example 48

Combination of 5 compounds with components containing sulphani!ic acid, ethanolamine, acetyl acetone & urea give buff ppt (BDJ).

### Example 49

Octa organic mixture containing S component is refluxed with benzaldehyde, acetophenone, urea &diosmin give yellow solid BDP.

### Example 50

Mixture containing T component is refluxed with nicotinic acid, pToluidine, Flavanone fused with sodium acetate form white ppt FB3.

### Example 51

Combination of T component with salicyaldehyde, benzamide in formaldehyde form yellow ppt DR.

Careful addition of nine organic substrate with suitable regent form auxiliaries is termed as A90M.

### Example 52

Combining one part of components containing benzoylglycine, phenyl hydrazine, urea, ethyl acetoacetate in glacial acetic acid is fused with another components containing benzamide, p-dibromobenzene, pheny! enediamine& m-nitro benzoic acid in a RB Flask stirred for 6 hrs at RT yellow ppt DEF.

### Example 53

Another nona organics containing part A consist of hippuric acid .ethylacetoacetate, pToluenesulphonamide, & Urea is agitated with another components containing formic acid, o hydroxyacetophenone, resorcinol, acetic acid in dimethyl formamide solvent giving white ppt HEI.

Combination of ten organic substrate under suitable conditions form chemical auxiliaries is termed as (A100M),

### Example 54

Decaorganic compounds containing U component, p-dibromobenzene, Sulphanilic acid, ethanolamine acetylacetone& urea is mixed with another components V containing o-chlorobenzldehyde, salicylic acid, 2.4 dihydroxyacetophenone, acetamide& formaldehyde yellow ppt JA.

### Example 55

U component in mix 1 of deca organic is carefully mixed with acetophenone, phenyl hydrazine, 2.4 dihydroxyacetophenone, o-chlorobenzaldehyde dark coloured crystal TF7.

### Example 56

Mixture containing benzyl chloride, thiophene 2-carboxylic acid, phenyl hydrazine, ethanolamine, acetophenone is carefully added to another R B Flask containing salicylic acid, acetamide, formaldehyde, m-nitroaniline, sulphanilic acid form yellow needle RTF7

From the examples 5-56, it is observed that fragment intricate functionalities through filtrate extension by creating auxiliary organic media so that filtrate library can be created. Reactions are homogenized in different conditions so that bond forming efficiency, transition of hit to lead, scouting to longer carbon atom compounds in the libraries, reducing hazardous waste, green approach, is looked in the technology so that new world of filtrate libraries are generated. Upstream and downstream of organic reactions consume bulky amount of solvent and quantity is significantly reduced in different reaction condition.

In other aspect any Lipophilicity of drug candidate in existing product is very less which can be counteracted through filtrate 2 filtrate technology. Where the process in the said technology is sustainable since environment, health, and safety metrics fit with financial goal of manufacturers. All the above factors are noticed in the existing products is transacted in our technology and showcased in biorganic to deca organic reaction filtrates

The invention has been described in an illustrative manner, and it is to be understood that the terminology which has been used is intended to be in the nature of words of description rather than of limitation.

In one aspect, the extended filtrate may be used for the next batch of filtrate reaction where filtrate extension reaction is carried on bilayer crystallization at room temperature. In this reaction the product is salted out and easily we can reduce the slurry wastages in a large quantity than conventionally.

### Example 57

The orthochloroaniline, and nv nitrobenzoic acid reacted along with hydroxylamine hydrochloride results filtrate orthochloroanilinium hydrochloride is the end product. It is recrystalised in the mother liquor as pure form.

### Example 58

The benzaldehyde and acetophenone are reacted along with urea which results that dibenzylhydrazine received as filtrate which promotes the Elimination of the reduction over catalyst, no nitrosation, no reduction over lithium aluminum and hydride catalyst, without the use of solvent ethanol the reaction is achieved. And no hydrogen atmosphere, no heating, no consumption of energy required. It is other object achieved here that the reaction occurs at normal condition, Larger difference between their operating condition and drawbacks does not exist with model reaction, environment, pH, and reaction rate.

### Example 59

The reaction between ethyl acetoacetate and urea is observed where the 4,6 dimethyl urea filtrate extension results Biginelli compounds as end product, this is achieved without thermal heating and re-cycling Ionic liquids Table 1: Docking energy of compounds against protein 3ACX (Antibacterial)

| | | | | |
|---|---|---|---|---|
| 10 | −1.341 | ASN168 | 1.834 | Conventional |
| | | | | Hydrogen |
| | | | | Bond |
| | | TYR41 | 3.22062 | Pi-Donor Hydrogen |
| | | | | Bond |
| | | TYR41 | 5.76051 | Pi-Pi Stacked |
| | | TYR41 | 5.08983 | Pi-Alkyl |
| | | TYR248 | 4.59947 | Pi-Alkyl |
| 11 | −0.847 | VAL133 | 2.87663 | Carbon Hydrogen |
| | | | | Bond |
| | | VAL133 | 3.02208 | Carbon Hydrogen |
| | | | | Bond |
| | | ASP48 | 3.42754 | Pi-Anion |
| | | ILE47.ASP48 | 4.30847 | Amide-Pi Stacked |
| | | ILE47 | 4.95133 | Pi-Alkyl |
| | | VAL133 | 5.1902 | Pi-Alkyl |
| 12 | 1.015 | ASN168 | 1.64769 | Conventional |
| | | | | Hydrogen |
| | | | | Bond |
| | | ARG171 | 2.3378 | Conventional |
| | | | | Hydrogen |
| | | | | Bond |
| | | TYR248 | 2.1998 | Conventional |
| | | | | Hydrogen |
| | | | | Bond |
| | | TYR245 | 2.04107 | Conventional |
| | | | | Hydrogen |
| | | | | Bond |
| 13 | 2.681 | ASP48 | 1.88751 | Salt Bridge; |
| | | | | Attractive Charge |
| | | ASP48 | 1.82732 | Salt Bridge; |
| | | | | Attractive Charge |
| | | ARG171 | 1.92383 | Conventional |
| | | | | Hydrogen |
| | | | | Bond |
| | | TYR248 | 1.83136 | Conventional |
| | | | | Hydrogen |
| | | | | Bond |
| | | VAL133 | 2.53662 | Carbon Hydrogen |
| | | | | Bond |
| | | TYR41 | 4.71298 | Pi-Alkyl |
| | | TYR245 | 5.1497 | Pi-Alkyl |
| | | TYR248 | 4.76877 | Pi-Alkyl |
| ciprofloxacin | −13.587 | | | |

TABLE 2
Docking energy of compounds against protein 1IYK (Antifungal)

| | | | | |
|---|---|---|---|---|
| 10 | −3.534 | VAL108 | 4.92089 | Pi-Alkyl |
| | | LEU415 | 4.94084 | Pi-Alkyl |
| 11 | −0.636 | GLY413 | 1.98265 | Carbon Hydrogen |
| | | | | Bond |
| | | CYS223 | 4.72003 | Pi-Sulfur |
| | | ARG224 | 2.34285 | Pi-Lone Pair |
| | | ARG224, | 2.81226 | Amide-Pi Stacked |
| | | TYR225 | | |
| | | VAL108 | 5.11261 | Pi-Alkyl |
| | | LEU415 | 4.05054 | Pi-Alkyl |
| | | LEU415 | 4.96684 | Pi-Alkyl |
| 12 | −3.307 | HIS227 | 2.84436 | Conventional |
| | | | | Hydrogen |
| | | | | Bond |
| | | HIS227 | 1.95515 | Conventional |
| | | | | Hydrogen |
| | | | | Bond |
| | | ASP412 | 1.83519 | Conventional |
| | | | | Hydrogen |
| | | | | Bond |
| | | GLY411 | 1.64528 | Carbon Hydrogen |
| | | | | Bond |
| 13 | −0.936 | ASP412 | 1.74199 | Salt Bridge; |
| | | | | Attractive Charge |
| | | ASP412 | 2.86402 | Attractive Charge |
| | | HIS227 | 3.01113 | Conventional |
| | | | | Hydrogen |
| | | | | Bond |
| | | HIS227 | 1.98751 | Conventional |
| | | | | Hydrogen |
| | | | | Bond |
| | | GLY411 | 2.68669 | Conventional |
| | | | | Hydrogen |
| | | | | Bond |
| | | GLY413 | 2.69104 | Conventional |
| | | | | Hydrogen |
| | | | | Bond |
| | | ASP412 | 2.73552 | Carbon Hydrogen |
| | | | | Bond |
| | | TYR225 | 3.55712 | Pi-Cation |
| | | CYS393 | 4.16733 | Alkyl |
| | | LEU394 | 4.91681 | Alkyl |
| | | TYR225 | 5.22997 | Pi-Alkyl |
| fluconazole | −15.125 | | | |

TABLE 3
Docking energy of compounds against
protein 1SA0 (Anthelmintic)

| | | | | |
|---|---|---|---|---|
| 10 | −13.263 | TYR385 | 5.23394 | Pi-Pi T-shaped |
| | | TRP387 | 5.75676 | Pi-Pi T-shaped |
| | | PHE518 | 5.72998 | Pi-Pi T-shaped |
| | | VAL434 | 5.60664 | Pi-Pi T-shaped |
| | | LEU507 | 5.10754 | Pi-Alkyl |
| | | LEU508 | 3.73554 | Pi-Alkyl |
| | | LEU384 | 5.00925 | Pi-Alkyl |
| | | MET522 | 4.8092 | Pi-Alkyl |
| 11 | −8.468 | TRP387 | 1.95063 | Conventional |
| | | | | Hydrogen |
| | | | | Bond |
| 12 | −13.073 | LEU352 | 2.19379 | Conventional |
| | | | | Hydrogen |
| | | | | Bond |
| | | GLN192 | 1.69965 | Conventional |
| | | | | Hydrogen |
| | | | | Bond |
| | | PHE518 | 2.8121 | Pi-Sigma |
| | | HIS90 | 4.29759 | Pi-Pi Stacked |
| | | PHE518 | 5.64429 | Pi-Pi T-shaped |
| | | VAL523 | 4.38032 | Alkyl |
| | | HIS90 | 4.83407 | Pi-Alkyl |
| | | ARG513 | 4.45212 | Pi-Alkyl |
| | | ALA515 | 5.27657 | Pi-Alkyl |
| | | VAL523 | 4.39395 | Pi-Alkyl |
| 13 | −13.263 | TYR385 | 5.23394 | Pi-Pi T-shaped |
| | | TRP387 | 5.75876 | Pi-Pi T-shaped |
| | | PHE518 | 5.72998 | Pi-Pi T-shaped |
| | | VAL434 | 5.60884 | Pi-Pi T-shaped |
| | | LEU507 | 5.10754 | Pi-Alkyl |
| | | LEU508 | 3.73554 | Pi-Alkyl |
| | | LEU384 | 5.00925 | Pi-Alkyl |
| | | MET522 | 4.8092 | Pi-Alkyl |
| Piperazine | −8.254 | | | |
| citrate | | | | |

TABLE 4
Decking energy of compounds against
protein 2OYE (Anti-inflammatory)

| | | | | |
|---|---|---|---|---|
| 10 | −12.442 | LEU384 | 1.89991 | Conventional |
| | | | | Hydrogen |
| | | | | Bond |
| | | MET522 | 4.53196 | Pi-Sulfur |
| | | TRP387 | 4.97386 | Pi-Pi T-shaped |
| | | MET522 | 3.39912 | Alkyl |
| | | ILE523 | 4.82963 | Alkyl |
| | | PHE518 | 3.28536 | Pi-Alkyl |
| | | LEU354 | 4.46837 | Pi-Alkyl |
| 11 | −17.803 | TRP387 | 1.94227 | Conventional |
| | | | | Hydrogen |
| | | | | Bond |
| | | LEU384 | 2.65214 | Carbon Hydrogen |
| | | | | Bond |
| | | ALA527 | 2.7471 | Pi-Donor Hydrogen |
| | | | | Bond |
| | | MET522 | 5.94618 | Pi-Sulfur |
| | | ALA527 | 5.03702 | Pi-Alkyl |
| | | LEU508 | 4.58873 | Pi-Alkyl |
| 12 | −12.208 | TRP387 | 1.83198 | Conventional |
| | | | | Hydrogen |
| | | | | Bond |
| | | LEU384 | 2.09327 | Conventional |
| | | | | Hydrogen |
| | | | | Bond |
| 13 | −11.017 | HIS386 | 2.71137 | Conventional |
| | | | | Hydrogen |
| | | | | Bond |
| | | TRP387 | 1.93338 | Conventional |
| | | | | Hydrogen |
| | | | | Bond |
| | | LEU384 | 1.98839 | Conventional |
| | | | | Hydrogen |
| | | | | Bond |
| | | LEU384 | 1.78832 | Conventional |
| | | | | Hydrogen |
| | | | | Bond |
| | | ILE523 | 2.97737 | Carbon Hydrogen |
| | | | | Bond |
| | | MET522 | 2.21327 | Carbon Hydrogen |
| | | | | Bond |
| | | MET522 | 3.04873 | Carbon Hydrogen |
| | | | | Bond |
| | | GLN383 | 2.9311 | Carbon Hydrogen |
| | | | | Bond |
| | | TYR504 | 2.92866 | Pi-Lone Pair |
| | | VAL451 | 4.48253 | Alkyl |
| | | PHE381 | 5.02495 | Pi-Alkyl |
| | | TYR385 | 5.05865 | Pi-Alkyl |
| | | HIS385 | 4.84723 | Pi-Alkyl |
| | | TRP387 | 5.06374 | Pi-Alkyl |
| | | TRP387 | 4.95036 | Pi-Alkyl |
| | | TYR504 | 4.7243 | Pi-Alkyl |
| Indomethacin | | | | |

TABLE 5
Docking results of Target molecules

| | Docking energy (Kcal/mol) | | | | |
|---|---|---|---|---|---|
| | | | | Anti- | |
| | Anti- | Anti- | | inflammatory | |
| | bacterial | fungal | Anthelmintic | COX-1 | COX-2 |
| | PDB ID: | POB ID: | PDB ID: | PDB ID: | PDB ID: |
| Compounds | 3ACX | 1IYK | 1SAO | 2OYE | 4COX |
| 10 | −1.341 | −3.534 | −13.263 | −12.442 | −17.355 |
| 11 | −0.847 | −0.836 | −8.468 | −17.803 | −19.415 |
| 12 | 1.015 | −3.307 | −13.073 | −12.208 | −16.223 |
| 13 | 2.651 | −0.838 | −13.263 | −11.017 | −17.169 |
| ciprofloxacin | −13.597 | | | | |
| Flucanazole | | −15.125 | | | |
| Piperazine | | | −8.254 | | |
| citrate | | | | | |
| Indomethacin | | | | −30.583 | −22.299 |

The scope of the invention is not to be construed as limited by the illustrative embodiments set forth herein, but is to be determined in accordance with the appended claims. Variations within the scope of the invention may be made by those ordinarily skilled in the art without departing from the essence of the invention as claimed herein.

1. An improved process to increase the performance of a reaction by filtration extension where the improvement in the process comprising of
a). obtaining filtrate 1 and filtrate 2 from the reactions of mixture 1 and mixture 2 respectively,
b). mixing the filtrate 1 and filtrate 2 in the next reaction,
Wherein the reaction condition is controlled by controlling pH of the mixture 1 and mixture 2, acid/base treatment, resulting plurality of filtrate extension chemical auxiliaries in higher level of organic media.

2. The improved process to increase the performance of a reaction as claimed in claim 1, wherein the filtrate 1 or filtrate 2 or both contain a molecular fragment

3. The improved process to increase the performance of a reaction as claimed in claim 1, wherein filtrate 1 or filtrate 2 is the transformed media from the initial stage

4. The improved process to increase the performance of a reaction as claimed in claim 1, wherein filtrate 1 or filtrate 2 is extended filtrate may be used for the next batch of filtrate reaction

5. The improved process to increase the performance of a reaction as claimed in claim 1, wherein the filtrate extension of a reaction may be performed by bilayer crystallization at room temperature thereby reducing the slurry wastages.
